(19) United States
(12) Patent Application Publication
(10) Pub. No.: US 2005/0059034 A1
(43) Pub. Date: Mar. 17, 2005

(54) Anonymous testing system and kit

(76) Inventors: Troy S. Tyler, New York, NY (US); Peter G. Siedler, New York, NY (US)

Correspondence Address: MORRISON & FOERSTER LLP, 1650 TYSONS BOULEVARD, SUITE 300, MCLEAN, VA, 22102, US

(21) Appl. No.: 10/763,408

(22) Filed: Jan. 26, 2004

Related U.S. Application Data
(60) Provisional application No. 60/442,126, filed on Jan. 24, 2003.

(51) Int. Cl. G06F 17/60; C12Q 1/68

(52) U.S. Cl. 435/6; 705/3

(57) ABSTRACT

Disclosed are methods and kits for conducting anonymous genetic testing. The methods and kits include provide a patient with an Alias ID and Password. The Alias ID is used to track a genetic sample from the patient. Both the Alias ID and Password are then used by the patient to obtain the results of the genetic test. The methods and kits allow a patient to have a genetic test performed while remaining anonymous.

### FIELD OF INVENTION

The present invention relates to methods and kits for conducting anonymous genetic testing. More particularly, the methods and kits allow a patient to obtain information about their genetics in an anonymous manner.

### BACKGROUND OF THE INVENTION

Historically, genetic diagnostic testing (DNA testing) has been limited to identifying conditions of serious medical importance. Access to and use of tests has, therefore, traditionally been limited to the medical and clinical communities. Recently, however, new tests have been developed that can be used to tremendously expand the information that individuals can learn about themselves. DNA tests now can be used for a variety of purposes including wellness, self-knowledge, and entertainment.

Individual consumers, who are taking an increasingly active role in gathering information and managing their own health-care choices, are interested in this information but are currently unable to access it.

Presently, there are two obstacles that stand in the way of wide spread DNA testing. First, while there are legitimate arguments as to why access to tests of serious medical importance should remain limited to the medical establishment, access to most tests should ultimately expand to consumers, through broader, less exclusive, and lower-cost distribution outlets (e.g. retail stores). Second, consumers have legitimate concerns about the potential uses—and misuses—of genetic test information. Accordingly, consumers need confidential access to simple DNA tests.

U.S. Patent Publication No. US 2002/0137025 A1 to Quattrocchi describes an anonymous testing system and a kit for obtaining a specimen from a patient. The kit can be used for taking a blood sample. In Quattrochi, a patient sends the blood sample to a laboratory using an assigned personal code. A patient obtains their results by telephone using only this personal code.

The method in Quattrochi is limited in that it fails to adequately secure the personal code. The patient's personal code is available to everyone who has access to the test kit and the patient's specimen. Accordingly, Quattrochi only offers the patient limited anonymity.

U.S. Pat. No. 4,777,964 to Briggs et. al. describes a kit for obtaining a blood sample. The kit allows a patient to obtain a blood sample and to mail the sample to a laboratory for testing. The results of the test are then mailed to the patient, allowing the patient to be tested without a personal appearance at a laboratory. However, Briggs et. al. fails to provide any anonymity to the patient. The patient's name and address are known to laboratory personnel handling the test.

Accordingly, a need exists for a secure method and kit for conducting anonymous genetic testing.

### SUMMARY OF THE INVENTION

Described are methods and kits for conducting anonymous genetic testing. The methods and kits allow a patient to acquire a genetic sample and to send the sample to a laboratory for testing while remaining anonymous.

One embodiment of the invention is an anonymous testing method. The method includes providing a patient with an Alias ID and a Password, obtaining a test sample from the patient identified only by the Alias ID, testing the sample to obtain a test result and providing the test results to the patient using the Alias ID and Password.

Preferably, the Alias ID includes six or more characters and the Password includes four or more characters. Preferably, the test is a DNA test and the sample is a DNA sample. The patient can be provided test results at a website. In addition to the test results, the patient can also be provided with recommendations based on the test results.

Another embodiment is a method of obtaining test results anonymously. The method includes obtaining an Alias ID and Password and providing a test sample for testing. The test sample is identified by Alias ID. The test results are obtained using the Alias ID and the Password.

Yet another embodiment is anonymous test kit that includes an Alias ID, a Password and instructions on obtaining a test sample.

### BRIEF DESCRIPTION OF THE DRAWINGS

The invention will be better understood by reference to the Detailed Description of the Invention when taken together with the attached Figures, wherein:

FIG. 1 is flowchart of one embodiment of a anonymous testing system in accordance with the invention;

FIG. 2 is a front exterior view of an anonymous testing kit in accordance with the invention;

FIG. 3 is a front interior view of the test kit of FIG. 2;

FIG. 4 is a front view of a test sample package included in the test kit of FIG. 2;

FIG. 5 is an interior view of a test sample package included in the test kit of FIG. 2;

FIG. 6 is a front view of a compact anonymous testing kit in accordance with the invention; and

FIG. 7 is a rear view of the compact anonymous testing kit of FIG. 6.

### DETAILED DESCRIPTION OF THE INVENTION

Described are systems and kits for anonymous genetic testing. The systems and kits allow a patient to acquire a test kit, collect a genetic sample, have the sample tested, obtain the results of the test and obtain additional information concerning the test that can include options and recommendations for possible treatments and product purchases, while the patient identity remains anonymous.

As used herein, the term “patient” is used to refer to the person that is getting their DNA tested. The patient need not have any specific medical condition or in need of any medical care. Further, a patient may not be getting their DNA tested for any medical related reason. For example, patients may have their DNA tested for only informational or entertainment purposes.

The term “test sample” refers to a sample provided by the patient to be tested. For DNA testing, a test sample can be anything containing the patient's DNA.

The test kit includes an Alias ID and a Password. Preferably, the test kit is sealed in such a way that tampering can be detected, for example, the package may be shrink-wrapped. The Alias ID is a unique patient identifier that allows the patient to be identified without personal identifying information such as the patients name and address. A Master Database containing the Alias ID and the Password that corresponds to the Alias ID is kept by the test provider.

The Alias ID can be any combination of numbers, letters and/or other characters. Preferably, the Alias ID contains a combination of at least six numbers, letters and/or other characters. More preferably at least seven numbers, letters and/or other characters. Most preferably at least ten numbers, letters and/or other characters.

The Password preferably contains at least four numbers, letters and/or other characters. More preferably, the Password contains at least six numbers, letters and/or other characters. Preferably, the Password is case sensitive.

Having both an Alias ID and Password allows the system to provide extra security and anonymity to patients. The DNA sample the patient supplies can be identified only by the Alias ID. Accordingly, when the patient sends their DNA sample to a laboratory for testing, the patient does not need to include the patient's Password, only the patients Alias ID. Accordingly, laboratory workers or other people who come in contact with the sample, such as physicians who may analyze the results, will not have both the Alias ID and Password.

The patient can be directed to collect a DNA sample or other sample by any known means using a provided sample collection kit. Preferred DNA samples include skin cell samples, blood samples and saliva samples. A more preferred DNA sample is a skin cell sample from the inside of the patient's cheek. The skin cells can be obtained from scraping the inside of the patient's cheek.

After the DNA sample has been supplied to a laboratory for testing, the test results listed by the patient's Alias ID, are supplied to a test provider. The test provider could be an entity that is part of the laboratory that is testing the patient's DNA or an entity separate from the laboratory. The test provider then links the patient's Alias ID to the patient's Password, using the Master Database, to allow the patient to gain access to the test results.

By providing a separate Alias ID and Password for each patient, this system provides extra security to the patient. The test provider is the only entity that the patient needs to contact to obtain the results. By making the contact between the test provider and the patient secure, for example through a secure website, the patient can be assured that they can obtain their test results without compromising their anonymity.

Further, the system allows the patient, if they choose, to provide personal information, such as their name, address, family history etc., to the test provider, without allowing access to this information to anyone else (for example laboratory or clinical personnel) that had access to their DNA sample and Alias ID.

The patient may wish to provide additional information to the test provider for a variety of reasons. For example, the test provider may be able to provide a more detailed analysis of the DNA results with more information, the test provider may also be able to provide the patient with nearby professionals that can help them (such as physicians), or send the patient a hardcopy of their test results in the mail. Further, the system allows the patient, if they choose, to allow outside third parties of their choosing (for example a family member or a physician) to access their information without allowing access to this information to anyone else (for example laboratory or clinical personnel).

The test kit can be acquired in a variety of different manners. The distribution stream that is chosen can depend upon the specific genetic testing that is to be performed. For example, the tests may be obtained by the patient from retail outlets such as pharmacies, grocery stores and vitamin stores.

The patient may also order a test kit from the manufacture or a distributor over the phone, through the mail or through an internet website. The test kit could then be mailed directly to the consumer.

This test kits can also be distributed by a number of professionals including doctors, lawyers, personal fitness instructors and nutritionists. For example, a doctor, personal fitness instructor, or nutritionist may distribute a health related test, for example an obesity propensity test, to a patient. In another example, a lawyer may distribute a paternity test to a client.

Although the identity of the patient in several of these distribution streams is not entirely anonymous, complete anonymity during distribution is not always important. This is because although the distributor may know who is purchasing a genetic test kit, at this point no one knows whether the purchaser will ultimately be the actual test taking patient nor does anyone know the actual results of the genetic test itself.

However, in some instances, complete anonymity is preferred. In one preferred distribution method, the test kits are distributed through an anonymous distribution channel that does not require that the patient or purchaser interact directly with any other person. For example, the test kits can be distributed through specially designed vending machines. By distributing the test kit through vending machines, the patient can purchase a test kit without having to reveal the fact that he is purchasing a genetic test to another person.

Another example of an anonymous distribution stream is a secure website. The patient, or other test purchaser, could log into a secure website and order a test to be mailed to their home address. The test kits could then be mailed to the patient in a package that does not indicate its specific contents.

A compact test kit can be widely distributed as a magazine insert, handbill, mass mailing, etc. The test kit could include an Alias ID and Password or the test kit could include instructions on obtaining an Alias ID and Password. If the test kits include an Alias ID and Password, the test kit may instruct the patient to go to a website or otherwise contact the test provider to activate the Alias ID and Password. Alternatively, the test kits may arrive with the Alias ID and Password activated. If the test kit does not include an Alias ID and Password, the test kit can provide instructions to the patient for obtaining an Alias ID and Password. For example, the test kit may instruct the patient to go to a website where they can purchase an Alias ID and Password.

The test kits could be used to market products to specific consumers. Companies such as cosmetic, drug and food companies may provide the test kits for advertising, soliciting or pre-qualifying potential customers. In one example, magazine inserts could be used to allow patients to order a test kit or the inserts could even include a test kit. The test kit could be for targeting consumers that have particular needs that are related to genetic information. For example, a cosmetic company could insert a test kit that allows the company to determine what types of cosmetics would be best suited for the patient. The cosmetic company could then offer the patient one or more cosmetics that are suited for the patient according to their genetic makeup. Alternatively, a vitamin company could test patients for nutritional needs and then offer the patient a vitamin formula that is custom designed for the patient's needs.

The test kits may be used to test for multiple genetic conditions. For example, the test kit may instruct the patient to check-off on an included form that indicates which genetic tests they want performed. Alternatively, the test kit may direct the patient to the test provider's website where they can research different tests and choose which tests are to be performed.

Once the patient is in possession of the test kit, the patient can retrieve a DNA sample using the test kit and send the DNA sample to a laboratory for testing. The laboratory that receives the DNA sample preferably receives the DNA sample and the Alias ID of the patient but not the patient's Password. Accordingly, the laboratory personnel do not receive any information that could be used to identify the patient by name.

The laboratory then processes the DNA sample by performing the DNA test(s) requested by the patient or specified by the test purchased by the patient. Once the laboratory obtains the results of the DNA test(s) the results are listed only by the patient's Alias ID. The test results listed only by Alias ID can then be provided to other people for further analysis and recommendations.

Once the tests results have been obtained, the test results are provided to the test provider who makes the test results available to a patient who has both the Alias ID and Password.

The test results can be made available to a patient in a variety of manners. Preferably, the test results are provided to the patient along with information related to the test results. For example, if the test identifies the patient as someone who is susceptible to high blood pressure, dietary recommendations could be provided along with the test results. If the test result identifies the patient as prone to skin cancer, the patient could be provided with information related to identifying different types of skin cancers, sun blocks that can be used to protect the patient from the sun and a list of doctors that the patient could contact to get further information.

The test results can be entered into a computer database. A computer program could then be used to automatically analyze the results, for example the computer program could identify specific gene sequences as being characteristic of certain traits, and then automatically provide additional information to the patient along with the test results.

The laboratory can then sends the test results to the test provider that makes the results available to the patient. The test provider can upload the data from the laboratory to a website or otherwise make the data available to the patient.

The DNA test results can be made available to the patient over a website. In this embodiment, the patient logs into a secure website using both their Alias ID and Password. The test results are then made available to the patient. The test results could be made available alone or together with additional information. The additional information can include links to one or more other websites that provide additional information related to the characteristics identified by the genetic test.

Preferably, the patient is provided with a report that provides the results of the genetic test and recommendations together. The report could be, for example, a customized web page on a website or a document that can be downloaded by the patient and viewed and printed while the patient is offline.

The DNA test results may also be made available to the patient over a telephone system. In this embodiment, the patient can enter their Alias ID and Password using the digits on their telephone without interacting with another human. The test results, and information related to the test results, can then be provided to the patient using a computer generated voice.

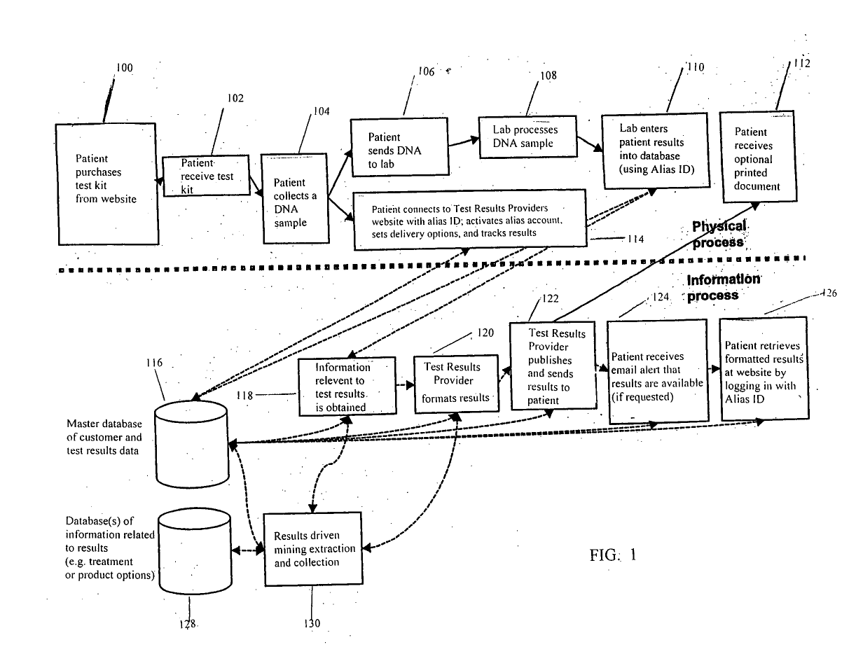

FIG. 1 is flowchart of one embodiment of an anonymous testing system. In FIG. 1 a patient first obtains a test kit at 100 from a test provider website. The test kit is then sent to the patient at 102. Using the kit, the patient next obtains a DNA sample at 104. The DNA sample is sent to a laboratory at 106 for DNA testing. The patient also uses the Alias ID and Password contained in the kit to log onto the test provider's website and to activate their at 114. On the test provider's website, the patient can choose how they want their test results delivered. The laboratory receives and processes the patient's DNA sample at 108. The laboratory enters the patient's test results into a database using the patient's Alias ID at 110.

The database with the patient's test results listed by their Alias ID is entered into the test provider's master database system 116. The test results can also be sent to another system(s) 128 for further analysis at 130. The analysis of the patient's test results is provided to the test provider's system at 118. The test results and any analysis/further information, is formatted to be provided to the patient at 120. If the patient agrees to receive the test results by mail, the system prints out the test results and sends them to the patient at 112. If the patient agrees to receive the results online at the test provider's system, the system can send an email alert to the patient (if the patient has provided an email address) notifying them that their test results are available at 124. The patient can then retrieve their formatted test results at the test provider's website at 126.

FIGS. 2-5 show one embodiment of a test kit according to the present invention. FIG. 2 shows the exterior package of the test kit 200. The exterior of the test kit includes a description 202 of the DNA test that will be performed. The exterior of the test kit 200 also can include information relevant to the sales channel being used to distribute the test kit. For example, if the test kit is to be distributed through a retail chain, the test kit may include UPC bar code and other sales information.

The Exterior of the test kit 200 is sealed in a manner that indicates whether the kit has been opened. For example, the test kit 200 may be sealed in shrink-wrap or enclosed in a sealed container that must be broken to open the test kit.

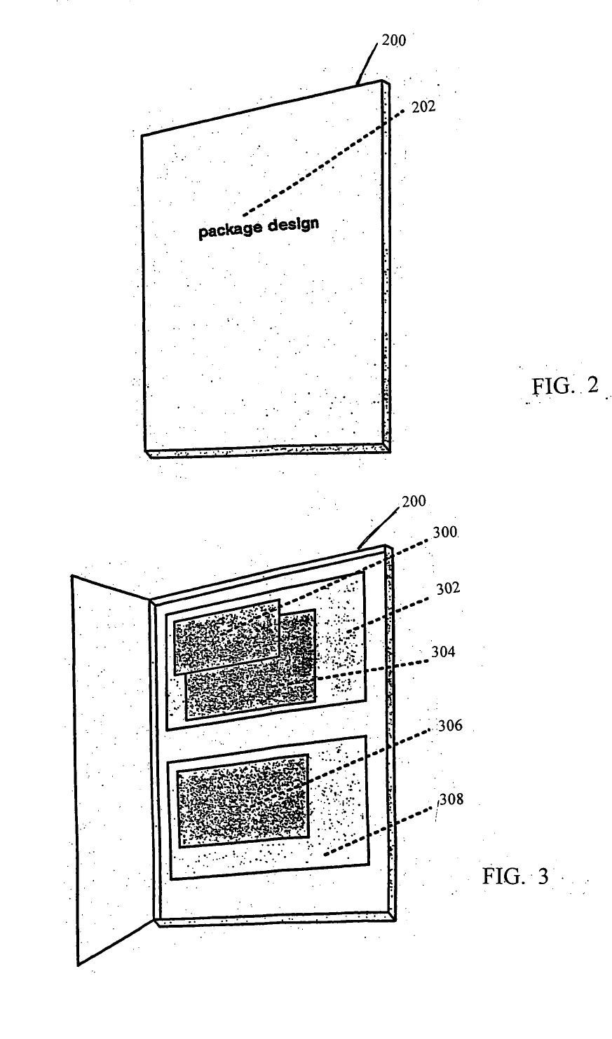

FIG. 3 shows the contents of the test kit 200. Inside test kit 200, is an instruction manual 300. The instruction manual 300 includes instructions for using the kit to obtain a DNA sample and for sending the DNA sample to a laboratory for testing. The instruction manual 300 also includes instructions for obtaining the results of the DNA test using the enclosed Alias ID and Password.

The kit may also include marketing materials 302. The marketing materials 302 describe the specific DNA test being performed and how the test results may be used by the patient.

A supplemental patient information form 304 can also be included in the test kit 200. Supplemental patient information form 304 is an optional form that can be filled out by the patient and returned to the test provider. By filling out and returning supplemental patient information form 304, the patient is able to obtain additional information about the test they are taking and can find out about additional DNA tests they can purchase.

The kit can also include a third party contact form 306. Third party customer contact form 306 is a form the patient can fill out to request information or order products from a third party related to the DNA test. For example, if the test is a test for providing a patient with obesity propensity test, the kit can include a form for contacting a nutritionist or diet center that can use the test results to help the patient control their weight.

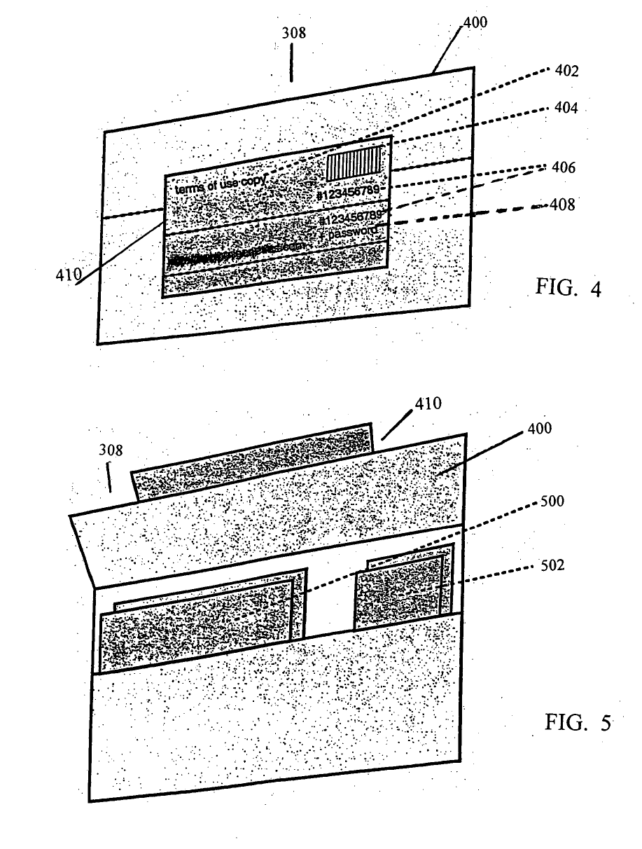

The kit also includes a test sample package 308. The test sample package 308 includes materials for collecting a DNA sample from a patient and materials for sending the collected DNA sample to a laboratory for processing. Test sample package 308 is described in greater detail in FIGS. 4 and 5.

FIG.4 shows a view of the test sample package 308 in its closed position. The test package 308 includes an envelope 400 containing the sample collection materials. Envelope 400 is sealed by label 410. Label 410 includes term of use information 402, barcode 404, Alias ID 406 and Password 408. Term of use information 402 can specify certain restrictions that the patient accepts by taking the DNA test. For instance the term of use information 402 can specify that the patient agrees that the DNA sample provided is their own DNA and that by opening envelope 400, the patient accepts the agreement. The upper portion of label 410 contains the patient's Alias ID 406 and a barcode version 404 of the Alias ID for tracking the patient's DNA sample.

The bottom portion of label 410 includes the patient's Alias ID and Password. The envelope 400 is opened by detaching the bottom portion of the label 410 from the envelope. The patient retains the bottom portion of the label. The Alias ID and Password, which together compromise the Alias ID token, on the bottom portion is used by the patient to obtain the results of the DNA test.

FIG. 5 shows the inside of test package 308. Inside test package 308 are sterile collection swabs 502 and sleeves 500. The sterile collection swabs 502 are used to scrape the inside of the patient's cheek to obtain a DNA sample. The used collection swabs 502 are then placed into sleeves 500. Sleeves 500 containing the used collection swabs 500 are then placed back into Envelope 400. Envelope 400 containing the patient's DNA material is then sent to a laboratory for testing.

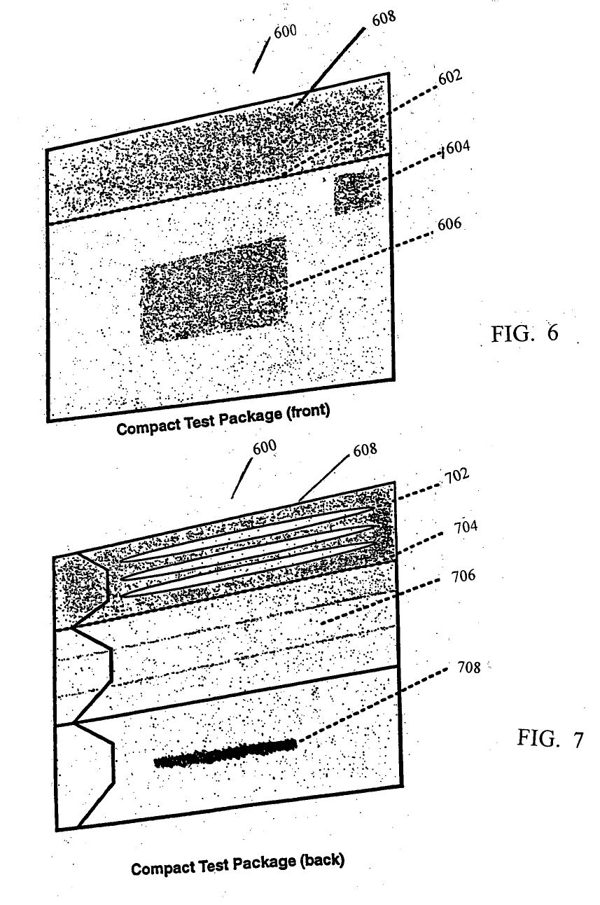

FIGS. 6 and 7 show a compact version 600 of the test package shown in FIGS. 4 and 5. FIG. 6 shows the front of the compact test package 600. The test package 600 includes prepaid postage 604 and the address 606 of the DNA laboratory that will test the patient's DNA. The top portion 608 of package 600 is a detachable pouch that contains sterile collection swabs 702. Partitioned portion 706 of package 600 contains partitioned envelopes for placing the swabs 702 in after they are used to collected DNA samples from the patient. The bottom portion 708 of the package 600 includes instructions for the patient on obtaining their DNA test results.

Once the patient has obtained a DNA sample using swabs 702 and placed the swabs 702 in partitioned portion 706. The package 600 is sealed and sent to a laboratory for testing. The compact test package 600 can be mailed to prospective clients or placed in magazines as an insert.

Following are six examples of how the anonymous testing system may be adminstered.

### EXAMPLE 1

An external customer contact (ECC), in this case, a third party therapeutic, health and wellness partner distributes a wellness test package (e.g. obesity propensity test).

Distribution

The partner includes the cost of the test package in the clinic program. The patient goes to a weight reduction clinic meeting where a counselor suggests that the patient take the obesity propensity test. After the patient agrees, the counselor breaks the seal on a carton containing a test kit.

Sample Collection

The patient cracks the seal on the sample collection package in the test kit after accepting the terms of use. The patient then reads the instructions and removes their unique Alias ID token containing their unique Alias ID and Password from the sample collection package. The patient then uses a sample collection swab contained in the sample collection package to obtain a DNA sample. The patient then places the sample collection swab in a swab container in the sample collection package. The swab container is then placed in a mailer that includes the patient's Alias ID. The mailer is then sent to a sent to a laboratory for testing.

Test Administration: Alias ID Registration

The patient goes to the test provider's website as indicated in the test kit. The patient enters their Alias ID and Password. The test provider's website activates the corresponding aliased account for that unique shrink-wrapped test kit.

Test Administration: Confidentiality Process and Tracking

Test directions instruct the patient to return to the website at a prescribed future time to access the test results the (patient may also monitor test-in-progress status—similar to package tracking.)

The laboratory receives the patient's DNA sample. The laboratory processes the sample and produces test results indexed only by Alias ID. The Lab sends the test results electronically to the test provider.

Results Delivery

The test provider formats the results for both electronic and physical delivery. The test provider publishes the test results at the patient's Password-protected aliased account on their website. The test provider mails the test results to those patients who have selected this delivery method.

The patient goes to the test provider's website, enters their unique Alias ID and Password and is then able to access their test results.

Results Mining

Based on the test results, information about additional clinic services may be provided to the patient. In addition, based on the test results, links to informational websites are also provided to the patient along with information on fitness equipment and weight reductions programs that can be used by the patient.

### EXAMPLE 2

A customer acquires a race/ancestry test through a test provider's website.

Distribution

A patient becomes aware of the test provider's product through their website. (Ad link, affiliated site, etc.). The patient purchases an ancestry test online. The patient supplies billing and shipment information through the secure website. The test provider then ships the test kit to the patient.

Sample Collection

Once the patient receives the test kit, the sample collection is the same as in Example 1.

Test Administration: Alias ID Registration

Same as Example 1.

Test Administration: Confidentiality Process and Tracking

Same as Example 1.

Test Administration: Processing and Tracking

Same as Example 1.

Results Delivery

Same as Example 1

Results Mining

Based on the test results, customized links to suggested media products are provided to the patient. The links can include links to various genealogical websites.

Example 3

A professional distributes a test package (e.g. paternity test) directly to one of their clients.

Distribution

The patient meets a lawyer at the lawyer's office. The lawyer breaks the seal on a carton containing the test kit and accepts the terms of use discussed with the patient. The patient “purchases” the test from Lawyer.

Sample Collection

The patient cracks opens the test kit by breaking a seal and accepting the terms of use. The lawyer fills out included forms and certifies the lawyer's identity. The lawyer retains the Alias ID token in the test kit that contains the Alias ID and Password.

The patient reads the instructions for obtaining a DNA sample. The patient then uses materials in the test kit (e.g. a sample collection swab) to capture a sample of patient DNA. The patient places the sample collection swab in a container, which is then placed in a mailer that includes the patient's Alias ID. The lawyer then mails the mailer to a laboratory for testing.

Test Administration: Alias ID Registration

The lawyer goes to a website as indicated on the Alias ID token. The lawyer enters the Alias ID and Password. The test provider then activates the corresponding aliased account for that unique shrink-wrapped paternity analysis test package.

Test Administration: Confidential Processing and Tracking

The test directions instruct the lawyer to return to the test provider's website at a prescribed future time to access the test results. Once the laboratory receives the patient's DNA sample, the laboratory processes the sample and produces test results indexed only by Alias ID. Lab sends test results electronically to the test provider.

Results Delivery

The test provider formats the test results and publishes the test results to the lawyer's Password-protected aliased account on their website. The lawyer goes to the website, enters the patient's unique Alias ID and Password and accesses the test results.

Results Mining

If the test taken by the patient is the non-legally binding version, the test provider up-sells the legal test based on the results obtained through the aliased web interface. If the patient purchased the product through a retail outlet (e.g. bookstore) the test results include local law firm referrals.

### EXAMPLE 4

A Retail partner (e.g. a retail pharmacy) distributes a customized drug matching test.

Distribution

The patient visits a retail pharmacy and purchases a customized drug matching test kit (with a physician's prescription, a pharmacist recommendation, or the patient sees an in store display). The patient then takes the test kit home.

Sample Collection

Same as Example 1. The test provider may allow multiple DNA tests to be taken using the single kit. The test process can deliver multiple results from a single Alias ID

Test Administration: Alias ID Registration

Same as Example 1.

Test Administration: Confidentiality Process and Tracking

Same as Example 1.

Test Administration: Processing and Tracking

Same as Example 1.

Results Delivery

Same as Example 1.

Results Mining

The test results include information about drug trial acquisition programs with links to join. The test results also provide dynamically served customized links to pharma-sponsored info and wellness programs.

### EXAMPLE 5

A patient acquires a customized vitamin test package from a test provider's store-in-store drop box located in a retail vitamin store.

Distribution

The patient goes to a retail vitamin outlet that has a store-in-store display for a vitamin test. The test provider's display describes the test benefits. The patient purchases (or receives for free) a shrink-wrapped test kit.

Sample Collection

The patient opens the test kit in store (or takes the test home). The patient cracks a seal on the test kit accepting the terms of use. The patient then reads included instructions and removes their unique Alias ID token from the test kit.

The patient uses materials in the test kit (e.g. a cheek swab) to capture a sample of DNA and prepares the DNA for mailing to a laboratory (or the patient drops the sample in an in-store drop-box). If the patient lacks internet access, or if the patient desires physical delivery of the test results, or if patient selects a non-confidential test option, the patient includes name and address information with the DNA sample.

Test Administration: Alias ID Registration

Same as Example 1.

Test Administration: Confidentiality Process and Tracking

Same as Example 1.

Test Administration: Processing and Tracking

Same as Example 1.

Results Delivery

Same as Example 1

Results Mining

The test results provide opportunities to buy customized vitamin programs and links to wellness programs in related fields. The patient may also receive gym and spa promotions.

### EXAMPLE 6

The patient acquires a multi-point generic test from a magazine insert. This Example shows a compact, free test kit for mass distribution of multiple tests and the supporting sample collection.

Distribution

The patient “discovers” a free blow-in insert or a compact give-away test kit in a magazine. The test kit includes multiple swabs for obtaining DNA samples. The test kit includes a website address to obtain information on using the kit.

Sample Collection

The patient goes to the website address included with the kit. The patient selects a desired test from the choices listed on the website. The patient then reads instructions on the website for taking the test. The patient agrees to the terms of use online and pays for the test. The website provides the patient with an Alias ID token that includes an Alias ID and a Password.

The patient then places their Alias ID in the sample mailer included with the kit. The patient uses materials in the kit to obtain a DNA sample and places the sample into the sample mailer. The mailer is then sent to a laboratory for testing.

Test Administration: Alias ID Registration

Same as Example 1.

Test Administration: Confidentiality Process and Tracking

Same as Example 1.

Test Administration: Processing and Tracking

Same as Example 1.

Results Delivery

Same as Example 1.

Results Mining

The test results provide dynamically served customized links to follow-up tests. The test results may also provide dynamically served customized magazine subscription suggestions.

Although the description refers to DNA testing, one skilled in the art would recognize that the principles described herein can also be applied to a variety of other applications where anonymity is desired.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

This application discloses numerical range limitations. Persons skilled in the art will recognize that the numerical ranges disclosed inherently support any range within the disclosed numerical ranges even though a precise range limitation is not stated verbatim in the specification because this invention can be practiced throughout the disclosed numerical ranges and at other numerical ranges which persons skilled in the art will find this invention operable.

1. An anonymous testing method comprising:
providing a patient with an Alias ID and a Password;
obtaining a test sample from the patient identified only by the Alias ID;
testing the sample to obtain a test result;
providing the test results to the patient using the Alias ID and Password.

2. The method of claim 1, wherein the Alias ID comprises six or more characters.

3. The method of claim 1, wherein the Password comprises four or more characters.

4. The method of claim 1, wherein the test sample is a DNA sample.

5. The method of claim 1, wherein the test is a DNA test.

6. The method of claim 1, wherein the patient is provided the test results at a website.

7. The method of claim 1, further comprising providing the patient with recommendations based on the test results.

8. A method of obtaining test results anonymously comprising:
obtaining an Alias ID and Password;
providing a test sample for testing, wherein the test sample is identified by Alias ID; and
obtaining test results using the Alias ID and the Password.

9. The method of claim 8, wherein the Alias ID comprises six or more characters.

10. The method of claim 8, wherein the Password comprises four or more characters.

11. The method of claim 8, wherein the test sample is a DNA sample.

12. The method of claim 8, wherein the test is a DNA test.

13. The method of claim 8, wherein the test results are obtained at a website.

14. The method of claim 13, further comprising obtaining recommendations based on the test results at the website.

15. An anonymous test kit comprising:
an Alias ID;
a Password; and
instructions on obtaining a test sample.

16. The anonymous test kit of claim 15, wherein the Alias ID comprises six or more characters.

17. The anonymous test kit of claim 15, wherein the Password comprises four or more characters.

18. The anonymous test kit of claim 15, wherein the test sample is a DNA sample.
